(19) United States
(12) Patent Application Publication
(10) Pub. No.: US 2007/0191331 A1
(43) Pub. Date: Aug. 16, 2007

(54) Crystalline forms of cefdinir potassium salt

(76) Inventors: Vinod Kumar Kansal, Haryana (IN); Dhirenkumar N. Mistry, Gujarat (IN); Saurabh Pandey, Uttar Pradesh (IN); Rakesh Patel, Gujarat (IN); Shlomit Wizel, Petah Tiqva (IL); Jean Hildesheim, Mazkeret Batya (IL)

Correspondence Address: KENYON & KENYON LLP, ONE BROADWAY, NEW YORK, NY, 10004, US

(21) Appl. No.: 11/591,217

(22) Filed: Oct. 31, 2006

Related U.S. Application Data
(60) Provisional application No. 60/732,001, filed on Oct. 31, 2005.

(51) Int. Cl. A61K 31/545; C07D 501/14

(52) U.S. Cl. 514/202; 540/222

(57) ABSTRACT

The present invention encompasses the solid state chemistry of cefdinir potassium salt.

### CROSS-REFERENCE TO RELATED APPLICATIONS

The application claims the benefit of priority to U.S. provisional Application Ser. 60/732,001, filed Oct. 31, 2005, hereby incorporated by reference.

### FIELD OF THE INVENTION

The present invention encompasses the solid state chemistry of cefdinir potassium salt.

### BACKGROUND OF THE INVENTION

Cefdinir is a third generation cephalosporin antibiotic for oral administration and has a broader antibacterial spectrum over general gram positive and gram negative bacteria than other antibiotics for oral administration. Cefdinir, currently marketed as OMNICEF®, is an antibiotic prescribed in a 300 mg oral capsule or a suspension of 125 mg/5 mL. OMNICEF® is prescribed for respiratory and ear infections. Cefdinir is 7-(Z)[2-(2-aminothiazol-4-yl)-2-hydroxyiminoacetimido]-3-vinyl-3-cephem-4-carboxylic acid and has the following structure:

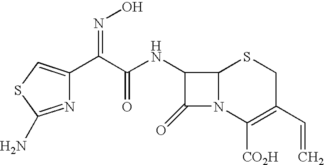

Examples 14 and 16 of U.S. Pat. No. 4,559,334 disclose the synthesis of cefdinir. In examples 14, cefdinir is obtained by reacting benzhydryl 7-(4-bromoacetoacetamido)-3-vinyl-3-cephem-4-carboxylate in dichloromethane and acetic acid with isoamyl nitrite at −3° C. to −5° C., followed by addition of acetylacetone. Thiourea was added and the benzyhydryl group was cleaved with trifluoroacetic acid. After work up, the organic layer was acidified and cooled at 0° C. to obtain the crystalline cefdinir. Compound 9 of example 2 discloses the sodium salt of cefdinir.

U.S. Pat. No. 4,935,507 discloses two methods of obtaining crystalline cefdinir. Crystalline cefdinir may be crystallized from methanol to obtain crystalline cefdinir Form A. Alternatively the Crystalline form is stepwise purified. In the stepwise process, the amorphous form was dissolved in water, washed with saturated sodium bicarbonate, acidified, passed by column chromatography, and treated with activated charcoal. The pH of the resultant solution was adjusted to 1.8 at 35° C. and the resultant crystalline cefdinir Form A was collected. The '507 patent shares one common inventor with the '334 patent and the same assignee. The '507 patent characterizes the product of examples 14 and 16 of the '334 patent as a crystalline like amorphous product, not a crystalline product. The '507 patent further states “the amorphous product has disadvantages that it is bulky, not so pure, unstable and insufficient in filtration rate, therefore it is not suitable for a pharmaceutical product or is not easy to handle in pharmaceutical preparations, in producing it in a large scale or in storage.”

PCT publication WO 98/45299 discloses a cefdinir dicyclohexylamine salt and that cefdinir may be purified via the dicyclohexylamine salt.

According to the PCT publication WO 02/098884, cefdinir is prepared by treating a cefdinir intermediate with a formic acid-sulfuric acid mixture or a formic acid-methanesulfonic acid mixture to obtain a crystalline salt of cefdinir and reacting the crystalline salt with a base in a solvent.

PCT publication WO 03/050124 describes a novel crystalline cefdinir potassium dihydrate, a process for its preparation and its use for the preparation of cefdinir.

US publication US 2004/0242556 discloses a crystalline form of Cefdinir, 7β-[(Z)-2-(2-amino-4-thiazolyl)-2-hydroxyimi-noacetamido]-3-vinyl-3-cephem-4-carboxylic acid, named crystal B, a process to prepare it and the use of cefdinir crystal B in pharmaceutical compositions.

The discovery of new polymorphic forms and solvates of a pharmaceutically useful compound provides a new opportunity to improve the performance characteristics of a pharmaceutical product. Also, cefdinir compositions often contain a high amount of impurities. There is a need in the art to prepare cefdinir with a desirable amount of purity.

### SUMMARY OF THE INVENTION

In one embodiment, the invention encompasses a crystalline form of cefdinir potassium salt (Form E) characterized by X-ray powder diffraction peaks at about 11.5, 12.3, 23.9, 24.6 and 27.0±0.2 degrees two-theta.

In another embodiment, the invention encompasses a process for preparing cefdinir potassium salt Form E comprising: combining cefdinir with water and H₃PO₄ to obtain a solution, combining the solution with a base and a source of potassium ions therewith to obtain a precipitate, and recovering the crystalline form.

In another embodiment, the invention encompasses a process for preparing cefdinir potassium salt Form E comprising dissolving cefdinir in a mixture of water, pyridine and an organic solvent, combining a base and a source of potassium ions therewith to obtain a precipitate, recovering the crystalline form, and drying it.

In another embodiment, the invention encompasses a process for preparing cefdinir potassium salt Form E comprising drying Cefdinir potassium salt form I.

In another embodiment, the invention encompasses a crystalline form of cefdinir potassium salt (Form I) characterized by X-ray powder diffraction peaks at about 8.5, 11.5, 16.7, and 18.4±0.2 degrees two-theta.

In another embodiment, the invention encompasses a process for preparing cefdinir potassium salt Form I comprising dissolving cefdinir in a mixture of water, pyridine and an organic solvent, combining the solution with a base and a source of potassium ions therewith to obtain a precipitate, and recovering the crystalline form.

In another embodiment, the invention encompasses a crystalline form of cefdinir potassium salt (Form J) characterized by X-ray powder diffraction peaks at about 8.3, 11.2, 11.5, 18.4, and 26.3±0.2 degrees two-theta.

In another embodiment, the invention encompasses a process for preparing the cefdinir potassium salt Form J comprising combining cefdinir, water and pyridine to obtain a reaction mixture, combining a base and a source of potassium ions therewith to obtain a precipitate, and recovering the crystalline form.

In another embodiment, the invention encompasses a crystalline form of cefdinir potassium salt (Form K) characterized by X-ray powder diffraction peaks at about 8.2, 11.1, 22.4, 23.7, 24.2, and 26.3±0.2 degrees two-theta.

In another embodiment, the invention encompasses a process for preparing the cefdinir potassium salt Form K comprising: combining cefdinir, water and an acid selected from the group consisting of: sulfuric acid, Formic acid, p-Toluenesulfonic acid, Trifluoroacetic acid, Methanesulfonic acid, and p-Toluenesulfonic acid (PTSA) to obtain a solution, combining a base and a source of potassium ions therewith to obtain a precipitate, and recovering the crystalline form.

In another embodiment, the invention encompasses a process for preparing the cefdinir potassium salt Form K comprising: combining cefdinir and water to obtain a suspension, combining a base and a source of potassium ions therewith to obtain a precipitate, and recovering the crystalline form.

In another embodiment, the invention encompasses a crystalline form of cefdinir potassium salt (Form L) characterized by X-ray powder diffraction peaks at about 12.9, 16.6, 19.8, 22.0, and 23.6±0.2 degrees two-theta.

In another embodiment, the invention encompasses a process for preparing cefdinir potassium salt Form L comprising drying cefdinir potassium salt Form K.

In another embodiment, the invention encompasses a crystalline form of cefdinir potassium salt (Form S) characterized by X-ray powder diffraction peaks at about 6.0, 9.7, 14.3, 15.3, 23.2, and 25.9±0.2 degrees two-theta.

In another embodiment, the invention encompasses a process for preparing cefdinir potassium salt Form S comprising dissolving cefdinir potassium salt in dimethyl acetamide, maintaining the obtained solution until a precipitate is formed, and recovering the crystalline form.

In another embodiment, the invention encompasses a crystalline form of cefdinir potassium salt (Form T) characterized by X-ray powder diffraction peaks at 6.0, 9.8, 14.4, and 21.1±0.2 degrees two-theta.

In another embodiment, the invention encompasses a process for preparing cefdinir potassium Form T comprising combining cefdinir potassium salt Form K with N-methyl pyrrolidone to obtain a precipitate, and recovering the crystalline form.

In another embodiment, the invention encompasses a process for preparing cefdinir comprising converting cefdinir potassium to cefdinir.

### BRIEF DESCRIPTION OF THE FIGURES

FIG. 1 illustrates an IR spectrum of cefdinir potassium salt Form K prepared according to Example 1.

FIG. 2 illustrates a PXRD pattern of cefdinir potassium salt Form K prepared according to Example 1.

FIG. 3 illustrates a PXRD pattern of cefdinir potassium Form K prepared according to example 7.

FIG. 4 illustrates a PXRD pattern of cefdinir potassium Form J.

FIG. 5 illustrates a PXRD pattern of cefdinir potassium Form E.

FIG. 6 illustrates a PXRD pattern of cefdinir potassium Form I.

FIG. 7 illustrates a PXRD pattern of cefdinir potassium salt Form S FIG. 8 illustrates a PXRD pattern of cefdinir potassium salt Form T.

FIG. 9 illustrates a PXRD pattern of cefdinir potassium salt Form L.

### DETAILED DESCRIPTION OF THE INVENTION

The present invention provides a process for preparation of cefdinir potassium on industrial scale. The potassium salts of the present invention allows for obtaining cefdinir with a desirable degree of purity, because due to the crystallization processes, the impurities and the side products are removed, so after converting the salt to cefdinir, higher purity profile is obtained.

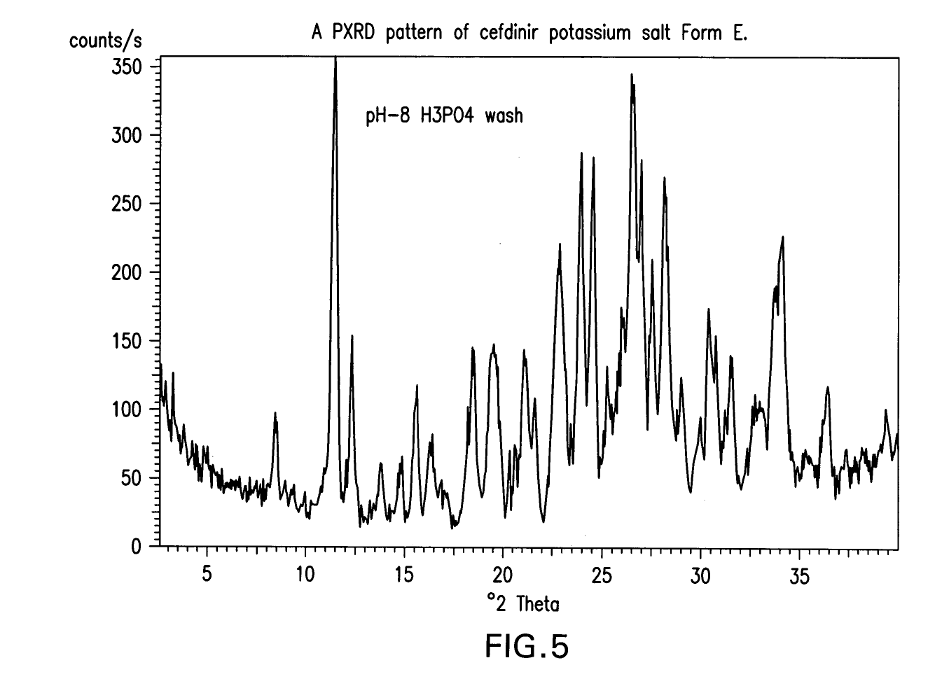

The present invention encompasses a crystalline form of cefdinir potassium salt, herein defined as Form E, characterized by X-ray powder diffraction peaks at about 11.5, 12.3, 23.9, 24.6 and 27.0±0.2 degrees two-theta. The crystalline form may be further characterized by X-ray powder diffraction peaks at about 8.5, 15.6, 18.5, 19.5, 21.0, 22.8, 26.6, 27.5 and 28.2±0.2 degrees two-theta. The crystalline form may be also substantially identified by the PXRD pattern depicted in FIG. 5. The crystalline form may be cefdinir hemi-pentahydrate characterized by a water content of about 10% water as determined by LOD values and TGA.

The invention encompasses a process for preparing Cefdinir potassium salt Form E comprising: combining cefdinir with water and H₃PO₄ to obtain a solution, combining the solution with a base and a source of potassium ions therewith to obtain a precipitate, and recovering the crystalline form. Preferably, prior to combining with the base and the source of potassium ions, the solution is heated to a temperature of about 30° C. to about 50° C., more preferably, about 40° C. Preferably, the base is added until obtaining a pH of about 7 to about 8, more preferably a pH of about 7 to about 7.5. Preferably, the base is also a source of potassium ions. Preferably, the base is an aqueous solution containing potassium carbonate or potassium bicarbonate. Preferably, base is added dropwise. The precipitate may be washed with water. The precipitate may be recovered by conventional techniques, such as filtration.

The invention encompasses a process for preparing Cefdinir potassium salt Form E comprising dissolving cefdinir in a mixture of water, pyridine and an organic solvent, combining a base and a source of potassium ions therewith to obtain a precipitate, recovering the crystalline form, and drying it. Preferably, the organic solvent is a C₁-C₄ alcohol, more preferably, the organic solvent is ethanol. Preferably, the base is added until obtaining a pH of about 7 to about 9, more preferably a pH of about 8. Preferably, the base is also a source of potassium ions. Preferably, the base is an aqueous solution containing potassium carbonate or potassium bicarbonate. Preferably, the base is added dropwise. The precipitate may be recovered by conventional techniques, such as filtration. The precipitate may be washed with water. The drying may be carried out at a reduced pressure and/or at high temperature to accelerate the drying process. Preferably, the drying is carried out at a pressure below about 100 mmHg. Preferably, the drying is carried out at a temperature of at least about 30° C., more preferably, at about 40° C.

The invention encompasses another process for preparing Cefdinir potassium salt Form E comprising drying Cefdinir potassium salt form I. Preferably, the drying is at a temperature of at least about 30° C., more preferably, at about 40°. Preferably, the drying is carried out at a pressure below about 100 mmHg.

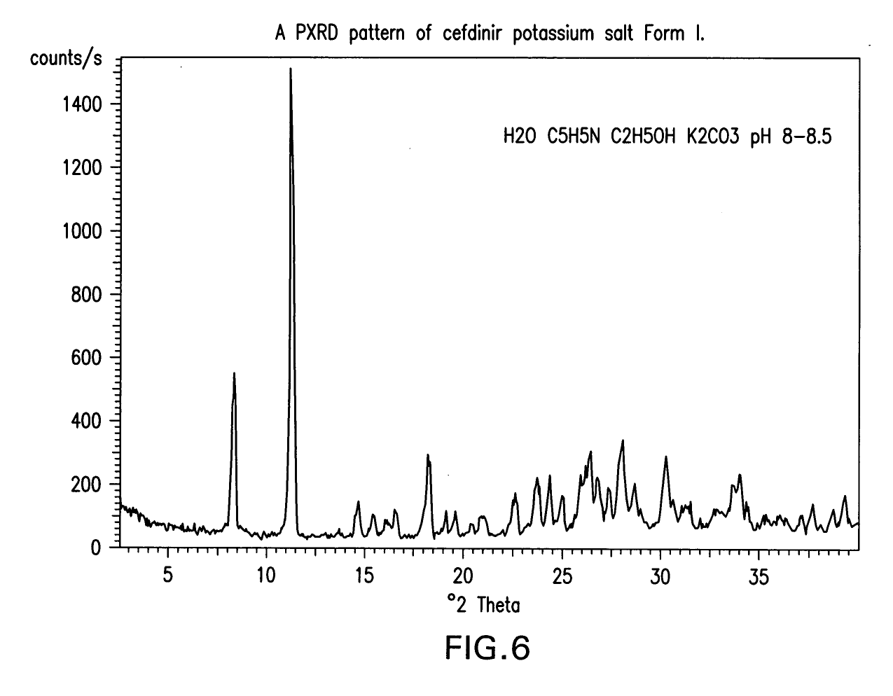

The present invention encompasses a crystalline form of cefdinir potassium salt, herein defined as Form I, characterized by X-ray powder diffraction peaks at about 8.5, 11.5, 16.7, and 18.4±0.2 degrees two-theta. The crystalline form may be further characterized by X-ray powder diffraction peaks at about 8.3, 24.9, 28.0, and 30.1±0.2 degrees two-theta. The crystalline form may be also substantially identified by the PXRD pattern depicted in FIG. 6.

The invention encompasses a process for preparing Cefdinir potassium salt Form I comprising dissolving cefdinir in a mixture of water, pyridine and an organic solvent, combining the solution with a base and a source of potassium ions therewith to obtain a precipitate, and recovering the crystalline form. Preferably, the organic solvent is a C₁-C₄ alcohol and more preferably the organic solvent is ethanol. Preferably, the base is added until obtaining a pH of about 7 to about 9, more preferably a pH of about 8. Preferably, the base is also a source of potassium ions. Preferably, the base is an aqueous solution containing potassium carbonate or potassium bicarbonate. The precipitate may be recovered by conventional techniques, such as filtration. The precipitate may be washed with water.

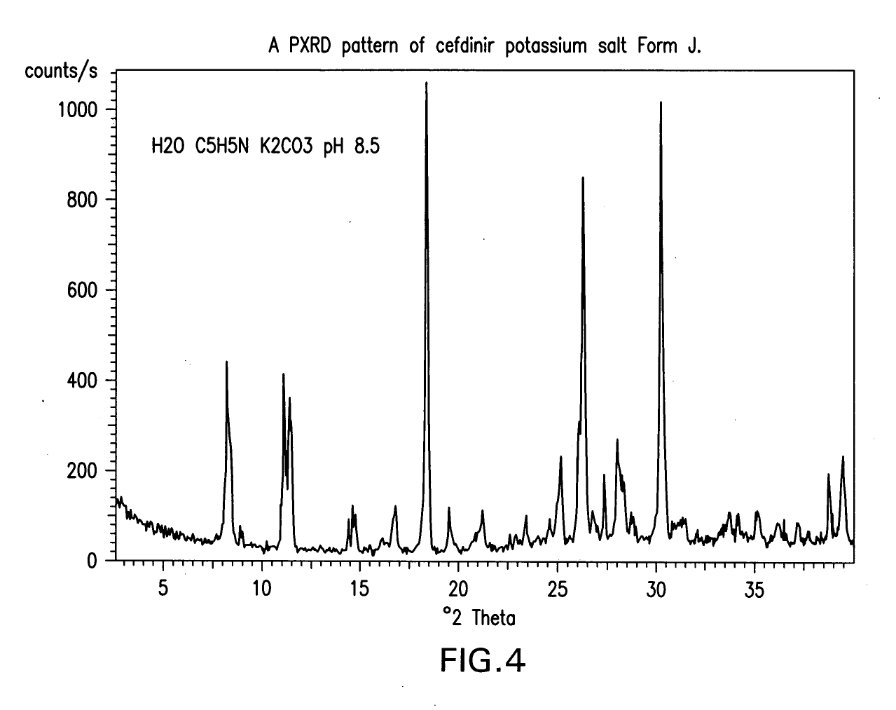

The present invention encompasses a crystalline form of cefdinir potassium salt, herein defined as Form J, characterized by X-ray powder diffraction peaks at about 8.3, 11.2, 11.5, 18.4, and 26.3±0.2 degrees two-theta. The crystalline form may be further characterized by X-ray powder diffraction peaks at about 19.5, 21.2, 25.2, 28.1, and 30.3±0.2 degrees two-theta. The crystalline form may be also substantially identified by the PXRD pattern depicted in FIG. 4.

The invention encompasses a process for preparing Cefdinir potassium salt Form J comprising combining cefdinir, water and pyridine to obtain a reaction mixture, combining a base and a source of potassium ions therewith to obtain a precipitate, and recovering the crystalline form. Preferably, the base is as described above.

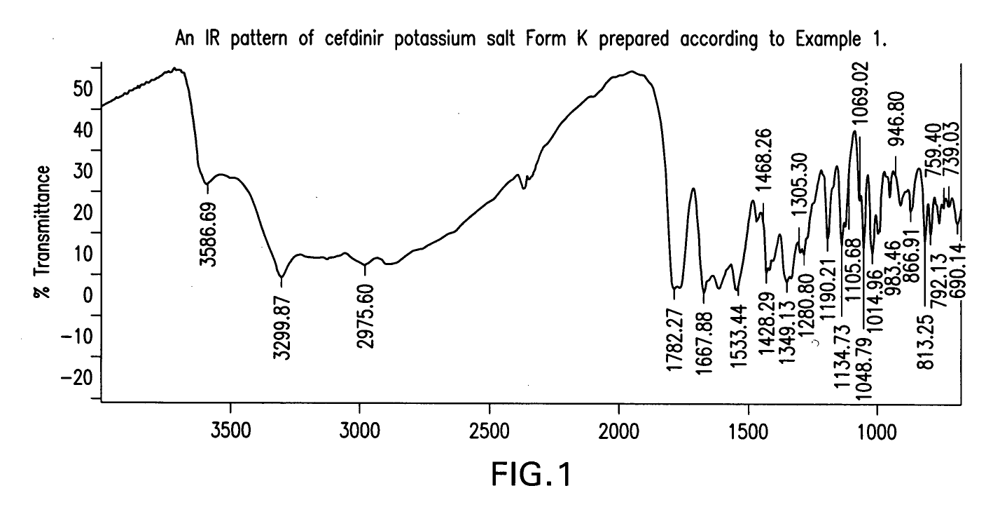

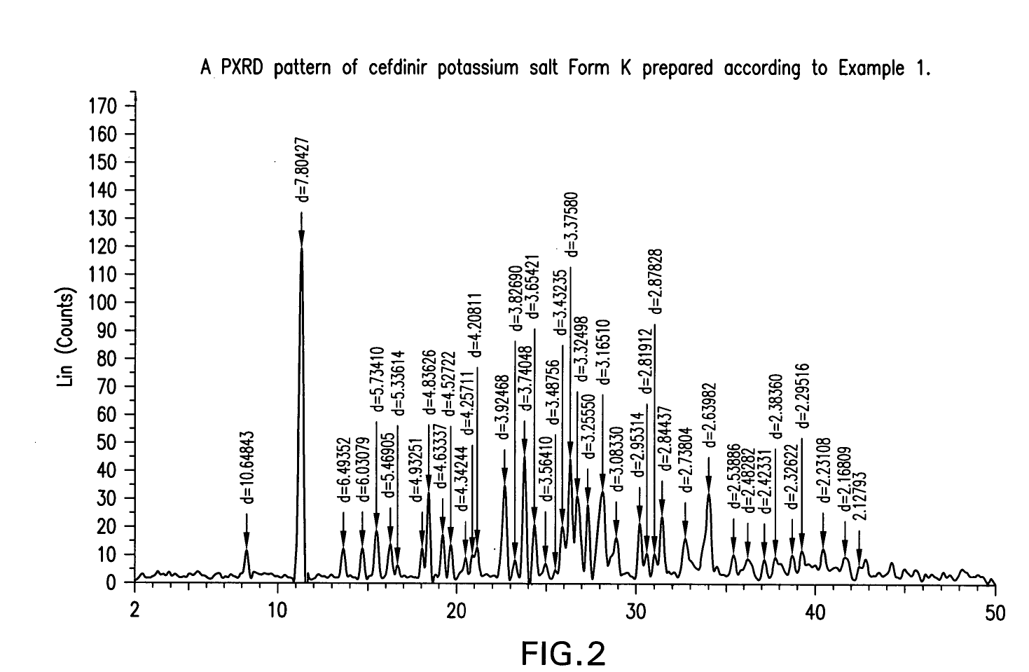

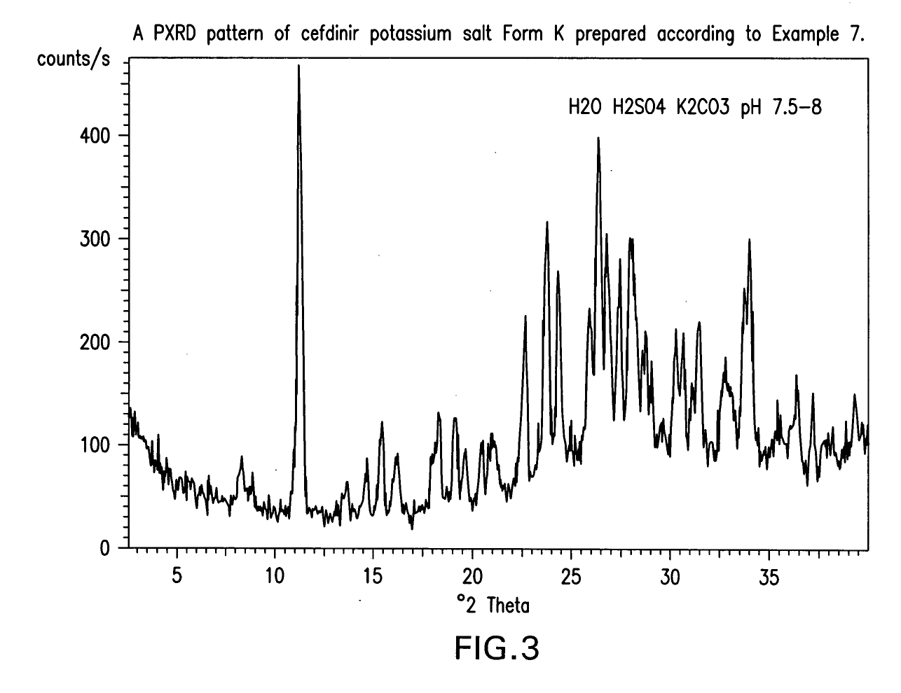

The present invention encompasses a crystalline form of cefdinir potassium salt, herein defined as Form K, characterized by X-ray powder diffraction peaks at about 8.2, 11.1, 22.4, 23.7, 24.2, and 26.3±0.2 degrees two-theta. The crystalline form may be further characterized by X-ray powder diffraction peaks at about 13.5, 14.5, 15.4, 16.1, 18.2, 19.5, 20.8, 26.7, and 27.3±0.2 degrees two-theta. The crystalline form may be also substantially identified by the PXRD pattern depicted in FIGS. 2 and 3. The crystalline form may be further characterized by an R spectrum with peaks at: 813, 1048, 1134, 1190, 1428, 1533 and 3299 cm⁻¹. The crystalline form may be further characterized by IR spectrum with peaks at: 690, 739, 759, 792, 866, 946, 983, 1014, 1069, 1105, 1280, 1305, 1349, 1468, 1667, 1782, 2975 and 3586 cm⁻¹. The crystalline form may be also substantially identified by the IR spectrum depicted in FIG. 1; The crystalline form may be characterized by a water content of about 10% to about 30% by weight. Preferably, the crystalline form may be characterized by a water content of about 13% to about 26% by weight, more preferably, by a water content of about 13% to about 15%, most preferably, by a water content of about 14.4% by weight, as measured by Karl Fisher analysis or by LOD.

The invention encompasses a process for preparing Cefdinir potassium salt Form K comprising: combining cefdinir, water and an acid selected from the group consisting of: sulfuric acid, Formic acid, p-Toluenesulfonic acid, Triflouroacetic acid, Methanesulfonic acid, and p-Toluenesulfonic acid (PTSA) to obtain a solution, combining a base and a source of potassium ions therewith to obtain a precipitate, and recovering the crystalline form. Optionally, a water miscible solvent is added to the solution. Preferably, the solvent is acetone. Preferably, the solution is heated to a temperature of at least about 40° C. Preferably, the base is added to obtain a pH of about 6 to about 9, more preferably, a pH of about 7.5 to about 8.5. Preferably, the reaction mixture with the base is cooled to a temperature of less than about 4° C. Preferably, the base is as described above. The precipitate may be recovered by conventional techniques, such as filtration. The precipitate may be washed with water. The precipitate may be dried. The pressure may be reduced or the temperature increased to accelerate the drying process. Drying may be carried out at a pressure below about 100 mmHg and a temperature of about 30° C. to about 50° C.

The invention encompasses a process for preparing Cefdinir potassium salt Form K comprising: combining cefdinir and water to obtain a suspension, combining a base and a source of potassium ions therewith to obtain a precipitate, and recovering the crystalline form. Optionally, a water miscible solvent is added to the suspension. Preferably, the water miscible solvent is acetone. Preferably, the suspension is heated to a temperature of at least about 40° C. Preferably, the base is added to obtain a pH of about 6 to about 9, more preferably, a pH of about 7.5 to about 8.5. Preferably, the reaction mixture with the base is cooled to a temperature of less than about 4° C. Preferably, the base is as described above. The precipitate may be recovered by conventional techniques, such as filtration. The precipitate may be washed with water. The precipitate may be dried. The pressure may be reduced or the temperature increased to accelerate the drying process. Drying may be carried out at a pressure below about 100 mmHg and a temperature of about 30° C. to about 50° C.

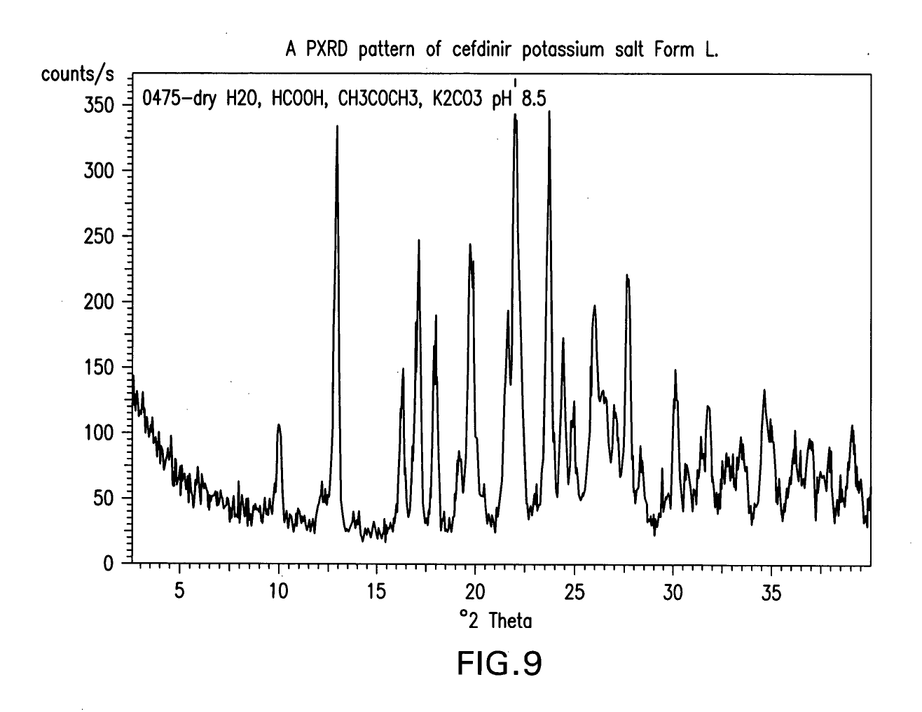

The present invention encompasses a crystalline form of cefdinir potassium salt, herein defined as Form L, characterized by X-ray powder diffraction peaks at about 12.9, 16.6, 19.8, 22.0, and 23.6±0.2 degrees two-theta. The crystalline form may be further characterized by X-ray powder diffraction peaks at about 9.9, 16.1, 17.8, 26.1, and 27.6±0.2 degrees two-theta. The crystalline form may be also substantially identified by the PXRD pattern depicted in FIG. 9.

The invention encompasses a process for preparing Cefdinir potassium salt Form L comprising drying cefdinir potassium salt Form K. Preferably, the drying is at a temperature of at least about 30° C., more preferably at about 40° C. Preferably, the drying is carried out at a pressure below about 10 mmHg.

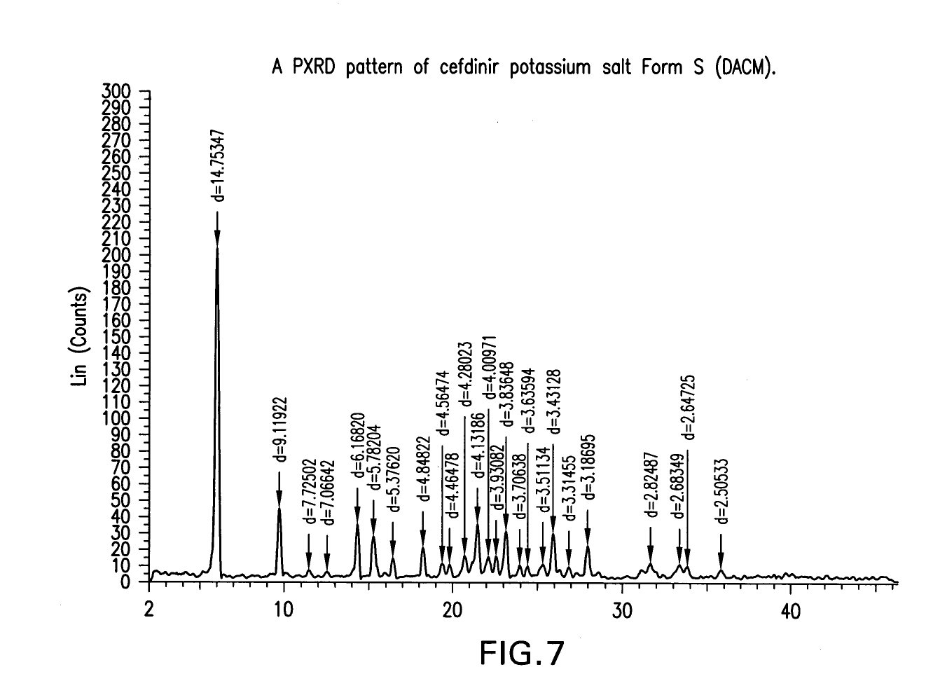

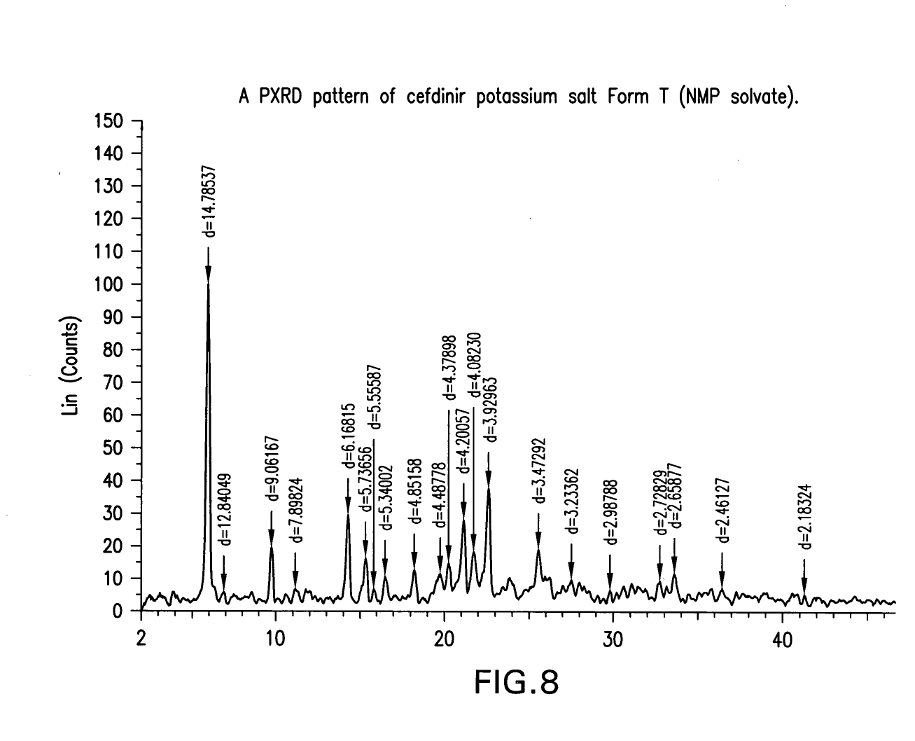

The present invention encompasses a crystalline form of cefdinir potassium salt, herein defined as Form S, characterized by X-ray powder diffraction peaks at about 6.0, 9.7, 14.3, 15.3, 23.2, and 25.9±0.2 degrees two-theta. The crystalline form may be a Dimethyl Acetamide (DMAC) solvate. Form S may be further characterized by X-ray powder diffraction peaks at about 16.5, 18.3, 21.5, 22.2, and 22.6±0.2 degrees two-theta. The crystalline form may be also substantially identified by the PXRD pattern depicted in FIG. 7.

The invention encompasses a process for preparing cefdinir potassium salt Form S comprising dissolving cefdinir potassium salt in dimethyl acetamide, maintaining the obtained solution until a precipitate is formed, and recovering the crystalline form. Preferably, the reaction mixture is at a temperature of about 25° C. to about 30° C. Preferably, the reaction mixture is stirred before the precipitate is formed. The precipitate may be washed with acetone. The precipitate may be dried. The pressure may be reduced or the temperature increased to accelerate the drying process. Drying may be carried out at a pressure below about 100 mmHg and a temperature of about 30° C. to about 50° C.

The present invention encompasses a crystalline form of cefdinir potassium salt, herein defined as Form T, characterized by X-ray powder diffraction peaks at 6.0, 9.8, 14.4, and 21.1±0.2 degrees two-theta. The crystalline form may be an N-methyl pyrrolidone (NMP) solvate. The crystalline form may be further characterized by X-ray powder diffraction peaks at 14.4, 15.4, 18.3, 21.8, and 22.6±0.2 degrees two-theta. The crystalline form may be also substantially identified by the PXRD pattern depicted in FIG. 8.

The invention encompasses a process for preparing Cefdinir potassium salt Form T comprising combining cefdinir potassium salt Form K with N-methylpyrrolidone to obtain a precipitate, and recovering the crystalline form. Preferably, the reaction is performed at a temperature of about 25° C. to about 30° C. Preferably, the reaction mixture is stirred before the precipitate is formed. The precipitate may be washed with acetone. The precipitate may be dried. The pressure may be reduced or the temperature increased to accelerate the drying process. Drying may be carried out at a pressure below about 100 mmHg and a temperature of about 30° C. to about 50° C.

The cefdinir used in the processes of the present invention may be prepared by the process provided in the examples or by using cefdinir made by any suitable process. Cefdinir used as starting material may be obtained by, for example, the processes described in U.S. Pat. Nos. 4,559,334, 4,870,168, 6,093,818, 7,105,659 or as described in WO 92/7840, these references are hereby incorporated by reference.

In another aspect of the invention, the potassium salt, in whatever polymorphic form, may be converted to cefdinir by use of an acid. A solution of the potassium salt may be prepared in water or a mixture of water and water-miscible organic solvent. Preferably, the solution of the potassium salt is prepared in water. Impurities from the solution may be removed by use of active carbon, a chelating agent and a filter. Due to substantial insolubility of cefdinir in water, the pH of the solution may be reduced to obtain the free acid and to precipitate cefdinir. A suitable pH is about 1 to about 4. Suitable acids include hydrochloric and sulfuric acids. Preferably, the acid is sulfuric acid. The temperature of the solution may also be decreased or the solution seeded to further induce crystallization. A suitable temperature is about 5° C. to about 15° C.

The potassium salt, in whatever polymorphic form, may also be converted to cefdinir according to U.S. Pat. No. 4,935,507.

The formation of the potassium salt can be incorporated in an industrial process for preparation of cefdinir, particularly in a synthetic process that reacts 7-amino-3-vinyl-3-cephem-4-carboxylic acid (7-AVNA, 0.4419 mol) with O-acetyl thioester.

In one aspect, the present invention provides a process for preparing cefdinir comprising by reacting a protected thioester of Formula I: 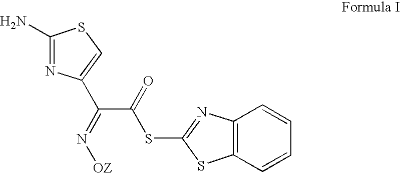
,7-amino-3-vinyl-3-cephem-4-carboxylic acid and a organic base in the presence of water and a water-miscible organic solvent to form protected cefdinir; converting the protected cefdinir to a cefdinir salt selected from the group consisting of cefdinir potassium and cefdinir cesium; and converting the cefdinir salt to cefdinir; wherein Z represents an oxime protecting group. In one embodiment, 7-amino-3-vinyl-3-cephem-4-carboxylic acid is reacted with O-acetyl thioester in a mixture of tetrahydrofuran and water while stirring the reaction mixture, the mixture is cooled to about 15-20° C., triethylamine is added to the reaction mixture to obtain a pH of about 8.0 to 8.2, methylene chloride is added to the reaction mixture while maintaining the temperature and stirring, water is added to the reaction mixture while maintaining the temperature and stirring, the organic and aqueous phases are separated, ammonium chloride is added to the aqueous phase to maintain the pH at about 7.8 to about 8.2, potassium carbonate solution is added to the aqueous phase, and the precipitated crystalline cefdinir potassium salt is obtained.

The present invention also encompasses pharmaceutical formulations comprising cefdinir of the present invention, and pharmaceutically acceptable excipient. Pharmaceutical compositions for administration to a mammal can be prepared from the cefdinir obtained by admixing said cefdinir with at least one pharmaceutically acceptable excipient. The pharmaceutical compositions are preferably administered orally for treatment or prevention of infections, particularly respiratory and ear infections.

The present invention further encompasses a process for preparing a pharmaceutical formulation comprising combining cefdinir of the present invention with at least one pharmaceutically acceptable excipient.

The present invention further encompasses the use of cefdinir of the present invention for the manufacture of a pharmaceutical composition.

Methods of administration of a pharmaceutical composition of the present invention can be administered in various preparations depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like.

Pharmaceutical compositions of the present invention can optionally be mixed with different forms of cefdinir and/or other active ingredients. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. EUDRAGIT®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, cefdinir and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol, and invert sugar may be added to improve the taste.

Preservatives and chelating agents such as alcohol, sodium benzoate, butylated hydroxy toluene, butylated hydroxyanisole, and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

When preparing injectable (parenteral) pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages can be conveniently presented in unit dosage form and prepared by any of the methods well-known in the pharmaceutical arts.

While the present invention is described with respect to particular examples and preferred embodiments, it is understood that the present invention is not limited to these examples and embodiments. The present invention, as claimed, therefore includes variations from the particular examples and preferred embodiments described herein, as will be apparent to one of skill in the art.

### EXAMPLES

The X-ray powder diffractions according to FIGS. 3-6, were performed on a Philips X-Ray Powder Diffractometer, Generator type: PW 1830, Goniometer type: PW 3020, Monochromator type: 1752/00, Cu target anode LFF was used. The scanning parameters were: 2-40 deg 2θ, at a rate: 3 deg./min.

The X-ray powder diffractions according to FIG. 2, were performed on a Siemens D 5000, Operating at 1.2 KV, Cu tube was used, the scanning parameters were: 2-50 deg. 2theta, step time: 0.5 sec.

FTIR was carried out by Nicolet, AVTAR, 370DTGS, manufactured by Thermoelectron. (IR of the potassium salt was carried out after preparing KBr palate.)

### Example 1

### Preparation of Crystalline Cefdinir Potassium Salt Form K

7-Amino-3-vinyl-3-cephem-4-carboxylic acid (“7-AVNA,” 100 g, 0.4419 mol) was added to tetrahydrofuran (1000 mL) followed by O-acetyl thioester (180 g, 0.4793 mol) and water (500 mL) with stirring. The reaction mass was cooled to 15° C. to 20° C. To this reaction mixture, triethylamine (62 mL) was added slowly at pH about 8.0-8.2. Stirring was continued and progress of the reaction was monitored by qualitative HPLC until 7-AVNA was less than 1%. At this stage methylene dichloride (1000 mL) was added and the reaction mixture was stirred for 15 min at 20° C. to 25° C. Water (1000 ml) was added to the reaction mass and stirred for 15 min at 20° C. to 25° C. The aqueous layer as separated and extracted with methylene chloride (500 mL). Thereafter, ammonium chloride (66 g) was added to the aqueous part in one lot at 20° C. to 25° C. and the pH was maintained between 8.0 to 8.2 by addition of 20% w/v aqueous potassium carbonate solution. The progress of reaction was monitored by qualitative HPLC until O-acetyl cefdinir was less than 0.5% by area. After completion of hydrolysis reaction, crystalline cefdinir potassium salts precipitated. Seeding may be necessary to precipitate cefdinir potassium salt. The mixture was stirred for one hour and thereafter, cooled to 5° C. to 110° C. and maintained at the temperature for one hour. The precipitate was collected by filtration and the crystals were washed with a solution of 1:1 acetone:water. The product was dried under atmospheric pressure until the moisture content was about 14.7% w/w. Cefdinir potassium salt Form K (135.2 g) was obtained in 99.0% purity (by HPLC).

### Example 2

### Preparation of Crystalline Cefdinir Potassium Salt Form K from Cefdinir

Cefdinir (10 g) was suspended in water (80 ml) at a temperature of 25° C. to 30° C. Potassium carbonate solution (40%) was added to adjust the pH to 8.0 to 8.2. After stirring the solution for 60 to 180 minutes, crystalline cefdinir potassium salt precipitated from solution. If necessary, the solution may be seeded with crystalline cefdinir potassium salt. The slurry was cooled to 5° C. to 110° C. and stirred for 60 minutes. The precipitate was collected, washed with acetone, and dried to obtain Cefdinir potassium salt Form K (7.5 g) HPLC (Purity>99.0%).

### Example 3

### Preparation of Crystalline Cefdinir from Crystalline Cefdinir Potassium

A. Preparation of Crystalline Cefdinir Form-A According to U.S. Pat. No. 4,935,507

Cefdinir potassium (15 g) obtained from the above examples (1 and 2) was dissolved in water (450 ml) at 25° C. to 30° C. The solution was treated with active carbon (1.5 g) and EDTA (0.15 g), and the mixture was stirred for 15-30 minutes. The solution was filtered through celite and the pH was adjusted to 1.8 to 2.4 by adding diluted sulphuric acid. A precipitate formed, was collected, and identified as crystalline cefdinir Form A (yield 11.3 g, HPLC 99.5%).

B. Preparation of Crystalline Cefdinir Form-B According to US 2003/204082 and US 2004/24556

Cefdinir potassium (15 g) obtained from the above examples (1 and 2) was dissolved in water (450 mL) at 25° C. to 30° C. The solution was treated with active carbon (1.5 g) and EDTA (0.15 g) and the mixture was stirred for 15-30 minutes. The solution was filtered through celite and the pH was adjusted to 1.8 to 2.4 by adding diluted sulphuric acid at 8° C. to 12° C. The solution was stirred and a precipitate was collected and identified as crystalline cefdinir Form-B (yield 11.3 g, HPLC 99.5%).

### Example 4

### Preparation of Crystalline Cefdinir Potassium Form I from Cefdinir

Water (25 mL), pyridine (10 mL) and ethanol (25 mL) were added to cefdinir (5 g). The pH was adjusted by adding potassium carbonate solution (2.5 ml of 40%) until the pH was 8. The precipitation formed was filtered and rinsed with water to obtain cefdinir Form I (2.5 g).

### Example 5

### Preparation of Crystalline Cefdinir Potassium Form J from Cefdinir

Water (25 mL) and pyridine (10 mL) were added to cefdinir (5 g). The pH was adjusted to 8.5 by the addition of potassium carbonate solution (2.8 ml of 40%). The precipitate was collected by filtration, rinsed with water, and dried to obtain cefdinir Form J (1.05 g) in wet form.

### Example 6

### Preparation of Crystalline Cefdinir Potassium Salt Form K from Cefdinir

Water (75 mL) and concentrated sulfuric acid (5 mL) were added to cefdinir (5 g). The pH was adjusted to 7 by the addition of potassium Carbonate solution (33 ml of 40%). The precipitate was collected by filtration, rinsed with water, and dried to obtain cefdinir Form K (2.8 g) in wet form.

### Example 7

### Preparation of Crystalline Cefdinir Potassium Salt Form K from Cefdinir

75 ml of water and 5 ml of concentrated sulfuric acid were added to 5 g of Cefdinir. The mixture was heated to 40° C. 33 ml of 40% Potassium Carbonate solution was added under stirring. The pH obtained was 7.5-8. The precipitation formed was filtered and rinsed with water. Cefdinir Potassium form K was obtained. (wet)

### Example 8

### Preparation of Crystalline Cefdinir Potassium Salt Form K from Cefdinir

75 ml of water and 5 ml of concentrated sulfuric acid were added to 5 g of Cefdinir. The mixture was heated to 40° C. 33 ml of 40% Potassium Carbonate solution was added under stirring. The pH obtained was 8-8.5. The precipitation formed was filtered and rinsed with water Cefdinir Potassium Form K was obtained. (wet)

### Example 9

### Preparation of Crystalline Cefdinir Potassium Salt Form K from Cefdinir

10 ml of water, 25 ml of Acetone, and 25 ml of Formic acid were added to 5 g of Cefdinir. The solution was continuously titrated with 98 ml of 40% Potassium Carbonate solution under stirring. The pH obtained was 8.5. The precipitation formed was filtered and rinsed with water. Cefdinir Potassium Form K was obtained. (wet)

### Example 10

### Preparation of Crystalline Cefdinir Potassium Salt Form K from Cefdinir

10 ml of water, 25 ml of Acetone, and 25 ml of Formic acid were added to 5 g of Cefdinir. The solution was continuously titrated with 95 ml of 40% Potassium Carbonate solution under stirring. The pH obtained was 6.5-7. The mixture was kept under refrigerating (4° C.) for 3 days. The precipitation formed was filtered and rinsed with water. Cefdinir Potassium Form K was obtained. (wet)

### Example 11

### Preparation of Crystalline Cefdinir Potassium Salt Form K from Cefdinir

10 ml of water, 25 ml of Acetone, and 25 ml of Formic acid were added to 5 g of Cefdinir. The solution was continuously titrated with 94 ml of 40% Potassium Carbonate solution under stirring. The pH obtained was 7.5-8. The precipitation formed was filtered and rinsed with water. Cefdinir Potassium Form K was obtained. (wet)

### Example 12

### Preparation of Crystalline Cefdinir Potassium Salt Form K from Cefdinir

25 ml of water and 15 ml of H₃PO₄ were added to 5 g of Cefdinir. The mixture was heated to 4° C. The solution was titrated with 59 ml of 40% Potassium Carbonate solution under stirring. The pH obtained was 6.5-7. The precipitation formed was filtered and rinsed with water. Cefdinir Potassium Form K was obtained. (wet)

### Example 13

### Preparation of Crystalline Cefdinir Potassium Salt Form K from Cefdinir

25 ml of water and 15 ml of H₃PO₄ were added to 5 g of Cefdinir. The mixture was heated to 40° C. The solution was titrated with 89.2 ml of 40% Potassium Carbonate solution under stirring. The pH obtained was 8.5-9. The precipitation formed was filtered and rinsed with water. Cefdinir Potassium Form K was obtained. (wet)

### Example 14

### Preparation of Crystalline Cefdinir Potassium Salt Form K from Cefdinir

5 ml of water were added to 5 g of Cefdinir. The mixture was heated to 40° C. 25 ml of 50% aqueous solution of p-Toluenesulfonic acid were added until complete dissolution. The solution was titrated with 10.6 ml of 40% Potassium Carbonate solution under stirring. The pH obtained was 8-8.5. The mixture was refrigerated (4° C.) overnight. The precipitation formed was filtered and rinsed with water. Cefdinir Potassium Form K was obtained. (wet)

### Example 15

### Preparation of Crystalline Cefdinir Potassium Salt Form K from Cefdinir

5 ml of water were added to 5 g of Cefdinir. The mixture was heated to 40° C. 25 ml of 50% aqueous solution of p-Toluenesulfonic acid were added until complete dissolution. The solution was titrated with 10.4 ml of 40% Potassium Carbonate solution under stirring. The pH obtained was 7.5. The mixture was refrigerated (4° C.) overnight. The precipitation formed was filtered and rinsed with water. Cefdinir Potassium Form K was obtained. (wet)

### Example 16

### Preparation of Crystalline Cefdinir Potassium Salt Form K from Cefdinir

5 ml of water were added to 5 g of Cefdinir. The mixture was heated to 40° C. 25 ml of 50% aqueous solution of p-Toluenesulfonic acid were added until complete dissolution. The solution was titrated with 10.6 ml of 40% Potassium Carbonate solution under stirring. The pH obtained was 8. The mixture was refrigerated (4° C.) overnight. The precipitation formed was filtered and rinsed with water. Cefdinir Potassium Form K was obtained. (wet)

### Example 17

### Preparation of Crystalline Cefdinir Potassium Salt Form K from Cefdinir

5 ml of water were added to 5 g of Cefdinir. The mixture was heated to 40° C. 25 ml of 50% aqueous solution of p-Toluenesulfonic acid were added until complete dissolution. The solution was titrated with 9.4 ml of 40% Potassium Carbonate solution under stirring. The pH obtained was 4.5. The mixture was refrigerated (4° C.) overnight. The precipitation formed was filtered and rinsed with water. A mixture of Cefdinir Form A and Cefdinir Potassium Form K was obtained. (wet)

### Example 18

### Preparation of Crystalline Cefdinir Potassium Salt Form K from Cefdinir

25 ml of water and 4.5 ml of Triflouroacetic acid were added to 5 g of Cefdinir. The mixture was heated to 40° C. The solution was titrated with 10.5 ml of 40% Potassium Carbonate solution under stirring. The pH obtained was 3.9-4.2. The mixture was refrigerated (4° C.) overnight. The precipitation formed was filtered and rinsed with water. A mixture of Cefdinir Form A and Cefdinir Potassium Form K was obtained. (wet)

### Example 19

### Preparation of Crystalline Cefdinir Potassium Salt Form K from Cefdinir

25 ml of water and 4 ml of Trifluoroacetic acid were added to 5 g of Cefdinir. The mixture was heated to 40° C. The solution was titrated with 9.2 ml of 40% Potassium Carbonate solution under stirring. The pH obtained was 7.5. The mixture was refrigerated (4° C.) overnight. The precipitation formed was filtered and rinsed with water. Cefdinir Potassium Form K was obtained. (wet)

### Example 20

### Preparation of Crystalline Cefdinir Potassium Salt Form K from Cefdinir

25 ml of water and 4 ml of Trifluoroacetic acid were added to 5 g of Cefdinir. The mixture was heated to 40° C. The solution was titrated with 10.6 ml of 40% Potassium Carbonate solution under stirring. The pH obtained was 8-8.5. The mixture was kept under refrigerating (4° C.) for overnight. The precipitation formed was filtered, rinsed with water and dried. Cefdinir Potassium Form K was obtained. (dry)

### Example 21

### Preparation of Crystalline Cefdinir Potassium Salt Form K from Cefdinir

25 ml of water were added to 5 g of Cefdinir. The mixture was heated to 40° C. 3 ml of Methanesulfonic acid were added. A complete dissolution was obtained. The solution was titrated with 9.9 ml of 40% Potassium Carbonate solution under stirring. The pH obtained was 8.5. The mixture was refrigerated (4° C.) overnight. The precipitation formed was filtered and rinsed with water. Cefdinir Potassium form K was obtained. (wet)

### Example 22

### Preparation of Crystalline Cefdinir Potassium Salt Form K from Cefdinir

25 ml of water were added to 5 g of Cefdinir. The mixture was heated to 40° C. 3 ml of Methanesulfonic acid were added. A complete dissolution was obtained. The solution was titrated with 8.4 ml of 40% Potassium Carbonate solution under stirring. The pH obtained was 4.5. The mixture was refrigerated (4° C.) overnight. The precipitation formed was filtered and rinsed with water. Cefdinir Potassium Form K was obtained. (wet)

### Example 23

### Preparation of Crystalline Cefdinir Potassium Salt Form K from Cefdinir

25 ml of water were added to 5 g of Cefdinir. The mixture was heated to 40° C. 3 ml of Methanesulfonic acid were added. A complete dissolution was obtained. The solution was titrated with 10.2 ml of 40% Potassium Carbonate solution under stirring. The pH obtained was 8.5. The mixture was refrigerated (4° C.) overnight. The precipitation formed was filtered and rinsed with water. Cefdinir Potassium Form K was obtained. (wet)

### Example 24

### Preparation of Crystalline Cefdinir Potassium Salt Form K from Cefdinir

25 ml of water were added to 5 g of Cefdinir. The mixture was heated to 40° C. 3 ml of Methanesulfonic acid were added. A complete dissolution was obtained. The solution was titrated with 9.9 ml of 40% Potassium Carbonate solution under stirring. The pH obtained was 8.5. The mixture was refrigerated (4° C.) for overnight. The precipitation formed was filtered and rinsed with water. Cefdinir Potassium Form K was obtained. (wet)

### Example 25

### Preparation of Crystalline Cefdinir Potassium Salt Form K from Cefdinir

25 ml of water and 4.5 ml of Trifluoroacetic acid were added to 5 g of Cefdinir. The solution was titrated with 8.4 ml of 40% Potassium Carbonate solution under stirring. The pH obtained was 5.5. The mixture was stirred for 2 hours. The mixture was refrigerated (4° C.) overnight. The precipitation formed was filtered and rinsed with water. Cefdinir Potassium Form K was obtained. (wet)

### Example 26

### Preparation of Crystalline Cefdinir Potassium Salt Form K from Cefdinir

25 ml of water and 3 ml of Methansulfonic acid were added to 5 g of Cefdinir. The solution was titrated with 6.4 ml of 40% Potassium Carbonate solution under stirring. The pH obtained was 5.5. The mixture was stirred for 2 hours. The mixture was refrigerated (4° C.) overnight. The precipitation formed was filtered and rinsed with water. A mixture of Cefdinir Form A and Cefdinir Potassium Form K was obtained. (wet)

### Example 27

### Preparation of Crystalline Cefdinir Potassium Salt Form K from Cefdinir

5 ml of water and 38.5 ml of PTSA were added to 5 g of Cefdinir at 50° C. The solution was titrated with 9 ml of 40% Potassium Carbonate solution under stirring. The pH obtained was 6-6.5. The mixture was stirred for 2 hours, and refrigerated (4° C.) overnight. The precipitation formed was filtered and rinsed with water. Cefdinir Potassium Form K was obtained. (wet)

### Example 28

### Preparation of Crystalline Cefdinir Potassium Form K and Form L from Cefdinir

5 ml of water were added to 5 g of Cefdinir. Then, 25 ml of formic acid, another 5 ml of water, and 25 ml of Acetone were added. The solution was titrated with 65.1 ml of 40% Potassium Carbonate solution, and stirred for 3 h. The pH obtained was 5.5. The mixture was refrigerated (4° C.) overnight. The precipitation formed was filtered, rinsed with water and dried at 40° C. under high vacuum. Cefdinir Potassium Form K was obtained for the wet product. Cefdinir Potassium Form L was obtained for the dry product.

### Example 29

### Preparation of Crystalline Cefdinir Potassium Form K and Form L from Cefdinir

25 ml of water and 15 ml of Phosphoric acid were added to 5 g of Cefdinir at 40° C. The solution was titrated with 35.1 ml of 40% Potassium Carbonate solution, and stirred for 2 h. The pH obtained was 5.5. The mixture was refrigerated (4° C.) overnight. The precipitation formed was filtered, rinsed with water and dried at 40oC under high vacuum. Cefdinir Potassium Form K was obtained for the wet product. Cefdinir Potassium Form L was obtained for the dry product.

### Example 30

### Preparation of Crystalline Cefdinir Potassium Form L from Cefdinir

12 ml of 85% formic acid solution were added to 5 g of Cefdinir. A clear solution was obtained. 12 ml of Acetone were added. The solution was titrated with 18.9 ml of 40% Potassium Carbonate solution. The pH obtained was 4. The mixture was refrigerated (4° C.). The precipitate formed was filtered, rinsed with water and dried at 40oC under high vacuum. Cefdinir Potassium Form L was obtained for the dry product.

### Example 31

### Preparation of Crystalline Cefdinir Potassium Form L from Cefdinir

10 ml of water and 25 ml of 85% formic acid solution were added to 5 g of Cefdinir. A clear solution was obtained. 25 ml of Acetone were added. The solution was titrated with 75 ml of 40% Potassium Carbonate solution. The pH obtained was 7. The mixture was refrigerated (4° C.) overnight. The precipitate formed was filtered, rinsed with water and dried at 40° C. under vacuum. Cefdinir Potassium Form L was obtained for the dry product.

### Example 32

### Preparation of Crystalline Cefdinir Potassium Form E from Cefdinir

25 ml of water and 15 ml of H₃PO₄ were added to 5 g of Cefdinir. The mixture was heated to 40° C. The solution was titrated with 72.5 ml of 40% Potassium Carbonate solution under stirring. The pH obtained was 7-7.5. The precipitate formed was filtered and rinsed with water. Cefdinir Potassium Form E was obtained. (wet)

### Example 33

### Preparation of Crystalline Cefdinir Potassium Form E and Form I from Cefdinir

25 ml of water, 10 ml of Pyridine, and 25 ml of C₂H₅OH were added to 5 g of Cefdinir. The solution was titrated with 2.7 ml of 40% Potassium Carbonate solution under stirring. The pH obtained was 8-8.5. The mixture was refrigerated (4° C.) for 3 days. The precipitation formed was filtered, rinsed with water and dried under high vacuum at 40° C. Cefdinir Potassium Form I was obtained for the wet product. Cefdinir Potassium Form E was obtained for the dry product.

### Example 34

### Preparation of Crystalline Cefdinir Potassium Form E and Form I from Cefdinir

25 ml of water, 10 ml of Pyridine, and 25 ml of C₂H₅OH were added to 5 g of Cefdinir. The solution was titrated with 2.4 ml of 40% Potassium Carbonate solution under stirring. The pH obtained was 7.5-8. The mixture was refrigerated (4° C.) for 3 days. The precipitation formed was filtered, rinsed with water and dried under high vacuum at 40° C. Cefdinir Potassium Form I was obtained for the wet product. Cefdinir Potassium Form E was obtained for the dry product.

### Example 35

### Preparation of Cefdinir Potassium Dimethyl Acetamide Solvate Form S from Cefdinir Potassium Salt

Cefdinir potassium salt (10 gm) obtained from example-1 was dissolved in Dimethyl acetamide (80 ml) at 25-30° C. while stirring. Crystalline cefdinir potassium salts solvate was crystallized out and filtered under nitrogen atmosphere. The product was washed with Acetone (100 ml) and dried under reduced pressure at 40⁰-45⁰ C for 4-5 hr. (Water: 2.1%, Yield: 0.92 w/w).

### Example 36

### Preparation of Cefdinir Potassium N-methylpyrollidone Solvate Form T from Cefdinir Potassium Salt

Cefdinir potassium salt (10 gm) obtained from example-1 was dissolved in N— methyl pyrollidone (80 ml) at 25-30° C. while stirring. Crystalline cefdinir potassium salts solvate was crystallized out and filtered under nitrogen atmosphere. The product was Wash with Acetone (100 ml) and dried under reduced pressure at 40⁰-45⁰ C for 4-5 hr. (Water: 1.8%, Yield: 0.75 w/w).

What is claimed is:

1. A crystalline form of cefdinir potassium salt (Form E) characterized by X-ray powder diffraction peaks at about 11.5, 12.3, 23.9, 24.6 and 27.0±0.2 degrees two-theta.

2. The crystalline form of claim 1 further characterized by X-ray powder diffraction peaks at about 8.5, 15.6, 18.5, 19.5, 21.0, 22.8, 26.6, 27.5 and 28.2±0.2 degrees two-theta.

3. The crystalline form of claim 1 substantially identified by the PXRD pattern depicted in FIG. 5.

4. The crystalline form of claim 1, wherein the crystalline form is a hemi-pentahydrate characterized by a water content of about 10% water as determined by LOD values and TGA.

5. A process for preparing cefdinir potassium salt of claim 1 comprising: combining cefdinir with water and H₃PO₄ to obtain a solution, combining the solution with a base and a source of potassium ions therewith to obtain a precipitate, and recovering the crystalline form.

6. The process of claim 5, wherein prior to combining with the base and the source of potassium ions, the solution is heated to a temperature of about 30° C. to about 50° C.

7. The process of claim 5, wherein the base is added until obtaining a pH of about 7 to about 8.

8. The process of claim 5, wherein the base is also a source of potassium ions.

9. The process of claim 5, wherein the base is an aqueous solution containing potassium carbonate or potassium bicarbonate.

10. A process for preparing cefdinir potassium salt of claim 1 comprising dissolving cefdinir in a mixture of water, pyridine and an organic solvent, combining a base and a source of potassium ions therewith to obtain a precipitate, recovering the crystalline form, and drying the crystalline form.

11. The process of claim 10, wherein the organic solvent is a C₁-C₄ alcohol.

12. The process of claim 11, wherein the organic solvent is ethanol.

13. The process of claim 10, wherein, the base is added until obtaining a pH of about 7 to about 9.

14. The process of claim 13, wherein the pH is 8.

15. The process of claim 10, wherein the base is also a source of potassium ions.

16. The process of claim 15, wherein, the base is an aqueous solution containing potassium carbonate or potassium bicarbonate.

17. The process of claim 10, wherein, the base is added dropwise.

18. The process of claim 10, wherein drying is carried out at a reduced pressure and/or at high temperature to accelerate the drying process.

19. The process of claim 10, wherein drying is carried out at a pressure below about 100 mmHg.

20. The process of claim 19, wherein drying is carried out at a temperature of at least about 30° C.

21. A process for preparing cefdinir potassium salt of claim 1 comprising drying Cefdinir potassium salt form I.

22. The process of claim 21, wherein the drying is at a temperature of at least about 30° C.

23. A crystalline form of cefdinir potassium salt (Form I) characterized by X-ray powder diffraction peaks at about 8.5, 11.5, 16.7, and 18.4±0.2 degrees two-theta.

24. The crystalline form of claim 23 further characterized by X-ray powder diffraction peaks at about 8.3, 24.9, 28.0, and 30.1±0.2 degrees two-theta.

25. The crystalline form of claim 23 as substantially identified by the PXRD pattern depicted in FIG. 6.

26. A process for preparing the crystalline form of claim 23 comprising dissolving cefdinir in a mixture of water, pyridine and an organic solvent, combining the solution with a base and a source of potassium ions therewith to obtain a precipitate, and recovering the crystalline form.

27. The process of claim 26, wherein the organic solvent is a C₁-C₄ alcohol.

28. The process of claim 27, wherein the organic solvent is ethanol.

29. The process of claim 26, wherein the base is added until obtaining a pH of about 7 to about 9.

30. The process of claim 26, wherein the base is also a source of potassium ions.

31. The process of claim 26, wherein the base is an aqueous solution containing potassium carbonate or potassium bicarbonate.

32. A crystalline form of cefdinir potassium salt (Form J) characterized by X-ray powder diffraction peaks at about 8.3, 11.2, 11.5, 18.4, and 26.3±0.2 degrees two-theta.

33. The crystalline form may of claim 32 further characterized by X-ray powder diffraction peaks at about 19.5, 21.2, 25.2, 28.1, and 30.3±0.2 degrees two-theta.

34. The crystalline form of claim 33 substantially identified by the PXRD pattern depicted in FIG. 4.

35. A process for preparing cefdinir potassium salt of claim 32 comprising combining cefdinir, water and pyridine to obtain a reaction mixture, combining a base and a source of potassium ions therewith to obtain a precipitate, and recovering the crystalline form.

36. A crystalline form of cefdinir potassium salt (Form K) characterized by X-ray powder diffraction peaks at about 8.2, 11.1, 22.4, 23.7, 24.2, and 26.3±0.2 degrees two-theta.

37. The crystalline form of claim 36, further characterized by X-ray powder diffraction peaks at about 13.5, 14.5, 15.4, 16.1, 18.2, 19.5, 20.8, 26.7, and 27.3±0.2 degrees two-theta.

38. The crystalline form of claim 37, wherein the crystalline form is substantially identified by the PXRD pattern depicted in FIGS. 2 and 3.

39. The crystalline form of claim 36 further characterized by an IR spectrum with peaks at: 813, 1048, 1134, 1190, 1428, 1533 and 3299 cm⁻¹.

40. The crystalline form of claim 36 further characterized by IR spectrum with peaks at: 690, 739, 759, 792, 866, 946, 983, 1014, 1069, 1105, 1280, 1305, 1349, 1468, 1667, 1782, 2975 and 3586 cm⁻¹.

41. The crystalline form of claim 36, substantially identified by the IR spectrum depicted in FIG. 1.

42. The crystalline form of claim 36, characterized by a water content of about 10% to about 30% by weight.

43. A process for preparing Cefdinir potassium salt of claim 36 comprising: combining cefdinir, water and an acid selected from the group consisting of: sulfuric acid, Formic acid, p-Toluenesulfonic acid, Triflouroacetic acid, Methanesulfonic acid, and p-Toluenesulfonic acid (PTSA) to obtain a solution, combining a base and a source of potassium ions therewith to obtain a precipitate, and recovering the crystalline form.

44. The process of claim 43, wherein a water miscible solvent is added to the solution.

45. The process of claim 44, wherein the solvent is acetone.

46. The process of claim 43, wherein the solution is heated to a temperature of at least about 40° C.

47. The process of claim 43, wherein the base is added to obtain a pH of about 6 to about 9.

48. The process of claim 43, wherein the reaction mixture with the base is cooled to a temperature of less than about 4° C.

49. The process of claim 43, further comprising drying at a pressure below about 100 mmHg and a temperature of about 30° C. to about 50° C.

50. A process for preparing cefdinir potassium salt of claim 36 comprising: combining cefdinir and water to obtain a suspension, combining a base and a source of potassium ions therewith to obtain a precipitate, and recovering the crystalline form.

51. The process of claim 50, wherein, a water miscible solvent is added to the suspension.

52. The process of claim 50, wherein, the water miscible solvent is acetone.

53. The process of claim 50, wherein the suspension is heated to a temperature of at least about 40° C.

54. The process of claim 50, wherein the base is added to obtain a pH of about 6 to about 9.

55. The process of claim 50, wherein the reaction mixture with the base is cooled to a temperature of less than about 4° C.

56. The process of claim 50, wherein drying is carried out at a pressure below about 100 mmHg and a temperature of about 30° C. to about 50° C.

57. A crystalline form of cefdinir potassium salt (Form L) characterized by X-ray powder diffraction peaks at about 12.9, 16.6, 19.8, 22.0, and 23.6±0.2 degrees two-theta.

58. The crystalline form of claim 57 further characterized by X-ray powder diffraction peaks at about 9.9, 16.1, 17.8, 26.1, and 27.6±0.2 degrees two-theta.

59. The crystalline form of claim 58, substantially identified by the PXRD pattern depicted in FIG. 9.

60. A process for preparing Cefdinir potassium salt of claim 57 comprising drying cefdinir potassium salt Form K.

61. The process of claim 60, wherein the drying is at a temperature of at least about 30° C.

62. The process of claim 60, wherein the drying is carried out at a pressure below about 10 mmHg.

63. A crystalline form of cefdinir potassium salt (Form S) characterized by X-ray powder diffraction peaks at about 6.0, 9.7, 14.3, 15.3, 23.2, and 25.9±0.2 degrees two-theta.

64. The crystalline form of claim 63, wherein the crystalline form is a dimethyl acetamide (DMAC) solvate.

65. The crystalline form of claim 63, further characterized by X-ray powder diffraction peaks at about 16.5, 18.3, 21.5, 22.2, and 22.6±0.2 degrees two-theta.

66. The crystalline form of claim 63, having a PXRD pattern as substantially identified by the PXRD pattern depicted in FIG. 7.

67. A process for preparing cefdinir potassium salt of claim 63 comprising dissolving cefdinir potassium salt in dimethyl acetamide, maintaining the obtained solution until a precipitate is formed, and recovering the crystalline form.

68. The process of claim 67, wherein the reaction mixture is at a temperature of about 25° C. to about 30° C.

69. The process of claim 67, wherein drying is carried out at a pressure below about 100 mmHg and a temperature of about 30° C. to about 50° C.

70. A crystalline form of cefdinir potassium salt (Form T) characterized by X-ray powder diffraction peaks at 6.0, 9.8, 14.4, and 21.1±0.2 degrees two-theta.

71. The crystalline form of claim 70, wherein the crystalline form is an N-methyl pyrrolidone (NMP) solvate.

72. The crystalline form of claim 70, further characterized by X-ray powder diffraction peaks at 14.4, 15.4, 18.3, 21.8, and 22.6±0.2 degrees two-theta.

73. The crystalline form of claim 70, substantially identified by the PXRD pattern depicted in FIG. 8.

74. A process for preparing cefdinir potassium of claim 70 comprising combining cefdinir potassium salt Form K with N-methylpyrrolidone to obtain a precipitate, and recovering the crystalline form.

75. A process for preparing cefdinir comprising converting cefdinir potassium to cefdinir.

76. The process of claim 75, wherein the cefdinir potassium is one of crystalline forms E, I, J, K, L S or T.

77. The process of claim 75, wherein the cefdinir potassium is prepared by reacting 7-amino-3-vinyl-3-cephem-4-carboxylic acid with O-acetyl thioester to obtain cefdinir and reacting the cefdinir with a base and a source of potassium to obtain cefdinir.

78. The process of claim 75, wherein the cefdinir potassium is converted to cefdinir by reaction with an acid.
